(19) United States
(12) Patent Application Publication
(10) Pub. No.: US 2008/0167705 A1
(43) Pub. Date: Jul. 10, 2008

(54) SHORT WIRE STENT DELIVERY SYSTEM WITH SPLITTABLE OUTER SHEATH

(75) Inventor: Charles W. Agnew, West Lafayette, IN (US)

Correspondence Address: BRINKS HOFER GILSON & LIONE/CHICAGO/COOK, PO BOX 10395, CHICAGO, IL, 60610, US

(73) Assignee: Cook Incorporated, Bloomington, IN (US)

(21) Appl. No.: 11/970,347

(22) Filed: Jan. 7, 2008

Related U.S. Application Data
(60) Provisional application No. 60/879,709, filed on Jan. 10, 2007.

(51) Int. Cl. A61F 2/84

(52) U.S. Cl. 623/1.12

(57) ABSTRACT

A delivery system and method for introducing a self-expanding prosthesis into a work site of a patient. The delivery system is introduced to the work site over an elongate guiding member. The delivery system is subsequently uncoupled from the guiding member upon deployment of the prosthesis while remaining within the work site. In an exemplary embodiment of the invention, the delivery system comprises an elongate outer member slidably disposed about an elongate inner member. A self-expanding prosthesis is disposed within the outer member and is laterally constrained thereby in a compressed delivery configuration, and is constrained against longitudinal movement relative to the inner member. The inner member further comprises a passageway extending through the distal portion thereof the passageway extending between a distal opening near the distal end of the inner member and a proximal opening spaced proximally from the distal end of the inner member. The outer member comprises an opening in communication with the proximal opening of the inner member. The inner and outer members are configured to allow a guiding member disposed through the passageway and openings to pass laterally out of the passageway and openings upon proximal movement of the outer member relative to the inner member and deployment of the prosthesis.

### CROSS-REFERENCE TO RELATED APPLICATIONS

This application claims the benefit of U.S. Provisional Application Ser. No. 60/879,709, filed Jan. 10, 2007, entitled “Short Wire Stent Delivery System With Splittable Outer Sheath”, the entire contents of which are incorporated by reference.

### TECHNICAL FIELD

The present invention relates to an apparatus for delivering an implantable prosthesis and, in particular, to an apparatus for introducing a self-expanding stent to a desired location within a bodily duct of a patient.

### BACKGROUND OF THE INVENTION

Self-expanding prosthesis, such as stents, artificial valves, distal protection devices, occluders, filters, etc., are used for a variety of applications. For example, self-expanding stents are used within the vascular and biliary tree to open strictures and maintain the patency of the vessel or duct. Current prosthesis delivery systems generally include an introducer catheter with a self-expanding prosthesis (e.g., a self-expanding stent) loaded within the distal end thereof. The introducer catheter maintains the stent in a compressed delivery configuration until the stent is positioned at the desired location within the patient. A pusher catheter is then used to deploy the stent from the distal end of the introducer catheter. In particular, the introducer catheter is retracted in a proximal direction relative to the pusher catheter to expose the stent from the distal end of the introducer catheter. As the stent is exposed from the distal end of the introducer catheter, it expands to engage the interior of the duct.

Current prosthesis delivery systems typically employ some form of minimally invasive surgery. In general, minimally invasive surgery is the practice of gaining access into a blood vessel, duct, or organ using a wire guide to facilitate the subsequent introduction or placement of catheters and other medical devices, and has been evolving since the Seldinger technique was first popularized during the late 1950s and 1960s. In the most basic form of the technique, a wire guide is introduced and advanced to a target site within the patient. A catheter or other medical device (e.g., prosthesis delivery system) having a lumen extending through the length thereof is then placed over and advanced along the wire guide. If a different catheter or medical device (e.g., a dilation balloon catheter or a second prosthesis delivery system) is required, then the first device is removed and exchanged for a second device. The second device is subsequently placed over and advanced along the wire guide to the target site. The Seldinger technique, also referred to as an ‘over the wire’ (OTW) or ‘long wire’ exchange technique, represented a significant advance by allowing an exchange of medical devices over a single indwelling wire guide without requiring displacement of the wire guide in the process and/or loss of access to the target site.

Although the ‘long wire’ or OTW technique still remains a commonly used method of exchanging devices in the vascular or biliary system, a technique was developed which allowed for a much shorter wire guide and more physician control over the wire. Variously known as the ‘rapid exchange,’ ‘monorail,’ or ‘short-wire’ technique, it differs from the OTW technique in that instead of the device being introduced over the length of the wire guide, the device is coupled to the wire guide for only a portion of the total length of the catheter device. More specifically, the wire guide is fed into and through a short lumen at the distal end of the catheter device, and then exits the lumen at a point between the catheter's distal and proximal ends via a port formed in the side of the catheter, which is typically located within the distal portion of the device. This allows the physician to have control of the proximal or external portion of the wire guide at all times as it exits the patient or scope and reduces the need for coordinating device movements with an assistant. When the coupled portion exits the patient (or endoscope in the case of gastroenterological or other endoscopic procedures), the physician performs a short exchange (instead of the traditional long-wire exchange) with a second device. To introduce the second device, the coupled portion of the catheter is advanced over the proximal end of the wire guide, while the physician is careful to maintain the wire in position so that its distal end is maintained within the work site and access is not lost.

Rapid exchange or short wire techniques have proven particularly desirable in coronary and vascular medicine whereby it is common to perform a sequence of procedures using multiple catheter-based devices over a single wire guide, such as prosthesis placement following angioplasty. Nevertheless, these techniques still require that a short exchange procedure be performed outside the patient, and care must be taken to prevent loss of wire guide access to the duct during the exchange procedure. Moreover, the process is further slowed by the frictional resistance between the wire guide and catheter, which remains a problem in subsequent exchanges as devices are advanced or retracted over the wire guide. Furthermore, existing devices do not offer the ability to place a second wire guide after the first one, such as to place stents in multiple ducts, since the catheter, which could otherwise serve as a conduit, must be removed from the patient and work site before the wire guide lumen could be made available for a second wire guide.

What is needed is an improved short-wire prosthesis delivery system and technique for efficiently and reliably introducing and exchanging devices within a work site which addresses one or more of the deficiencies described above.

### SUMMARY OF THE INVENTION

The foregoing problems are solved and a technical advance is achieved by an illustrative short wire prosthesis delivery system and method for introducing an expandable prosthesis over an indwelling guiding member, such as a wire guide, within a patient by remotely uncoupling the delivery system from the guiding member within the work site (defined as a lumen, duct, organ, vessel, other bodily passage or cavity, or the pathway leading thereto in which wire guide/guiding member access is maintained throughout a particular procedure or series of procedures), thereby facilitating the removal of the delivery system and simplifying introduction of a secondary access device or delivery system over the indwelling wire without an exchange of devices taking place outside of the patient. While the primary focus of this application is directed prosthesis (e.g., stent) delivery systems within the vascular system, the system and method of remote uncoupling of the delivery system within a work site can be adapted for any part of the body to perform any suitable procedure where the introduction, uncoupling and exchange of medical devices takes place over an indwelling guiding member. Examples include, but are not limited to the introduction and placement of stents, grafts, occluders, filters, distal protection devices, prosthetic valves, or other devices into the vascular system, including the coronary arteries, peripheral arterial system (e.g., carotid or renal arteries), or venous system (e.g., the deep veins of the legs). Other exemplary sites include the pancreatobiliary system or elsewhere in the gastrointestinal tract, the genito-urinary system (e.g., bladder, ureters, kidneys, fallopian tubes, etc.), and the bronchial system. Additionally, the present delivery system and method can be used for delivering prosthesis and other devices within body cavities, e.g., the peritoneum, pleural space, pseudocysts, or true cystic structures, via percutaneous placement and exchange through a needle, trocar, or sheath.

According to a first aspect of the present invention, the prosthesis delivery system comprises an elongate outer member slidably disposed about an elongate inner member. An expandable prosthesis is disposed within the outer member and is laterally constrained thereby in a compressed delivery configuration. The prosthesis is disposed about the inner member and is constrained against longitudinal movement relative thereto. The inner member further comprises a lumen or passageway extending through the distal portion thereof, the passageway extending between a distal opening near the distal end of the inner member and a proximal opening spaced proximally from the distal end of the inner member. The outer member comprises an opening in communication with the proximal opening of the inner member. The inner and outer members are configured to allow a guiding member disposed through the passageway and openings to pass laterally out of the passageway and openings upon proximal movement of the outer member relative to the inner member and deployment of the prosthesis.

In a preferred embodiment of the present invention, the outer member comprises a splittable wall between the distal end thereof and the opening, wherein the splittable wall is configured to separate upon proximal movement of the outer member relative to the inner member. In an exemplary embodiment, the splittable wall is separated by a guiding member extending outwardly from the proximal opening of the inner member and through the opening of the outer member. In another exemplary embodiment, the splittable wall is separated by one or more protrusions extending outwardly from an exterior surface of the inner member.

In the preferred embodiment of the present invention, the inner member comprises a channel in communication with the passageway and extending between the proximal and distal openings of the inner member. At least a portion of the channel is moveable from a first configuration to a second configuration upon proximal movement of the outer member relative to the inner member, wherein the guiding member is prevented from laterally passing through the channel when in the first configuration and is not prevented from laterally passing through the channel when in the second configuration. More specifically, the guiding member is laterally constrained within the passageway when the delivery system is in the delivery configuration, but is allowed to laterally exit the passageway when the delivery system is in the deployment/deployed configuration.

In an exemplary procedure, the above-described prosthesis delivery system is delivered to the target site within the patient by advancing the delivery system over a previously placed guiding member, such as a wire guide. In particular, the proximal end of the guiding member is inserted into the distal opening and through the passageway of the inner member, and then out through the proximal opening of the inner member and the opening in the outer member. The delivery system is then advanced along the guiding member until the prosthesis is positioned at the target location within the patient, e.g., at a stricture with the patient's vasculature system. The outer member is then retracted in a proximal direction relative to the inner member so as to deploy or expose the prosthesis from the distal end of the outer member, whereby the exposed prosthesis is allowed to expand. As the outer member is retracted, the portion of the wall of the outer member distal to the opening separates to allow the outer member to move proximally past the portion of the guiding member extending outwardly from the proximal opening of the inner member and through the opening in the outer member. In addition, as the outer member is retracted, the channel along the inner member and in communication with the passageway is allowed to open sufficiently to permit the guiding member to pass laterally out of the passageway and separate from the inner member. More specifically, proximal movement of the outer member relative to the inner member simultaneously results in: 1) deployment of the prosthesis; 2) separation of the guiding member from the outer member; and 3) separation of the guiding member from the inner member.

Once the prosthesis has been deployed and the guiding member has been separated from the inner and outer members, then the delivery system can be retracted and removed from the patient without dislodging or disrupting the position or placement of the guiding member. Removal of the delivery system is greatly facilitated by the elimination of friction which would have otherwise existed between the guiding member and the delivery system if the guiding member was still disposed through the passageway of the inner member. The guiding member may then be used for the introduction of another access device or delivery system into the patient. In some embodiments of the delivery system, the inner member of the delivery system may be left in the patient and used to introduce a second guiding member.

### BRIEF DESCRIPTION OF THE DRAWING

Embodiments of the present invention will now be described by way of example with reference to the accompanying drawings, in which:

FIG. 1 depicts a partial sectional view of an exemplary embodiment of a prosthesis delivery system in accordance with the present invention;

FIGS. 2-4 illustrate the distal portion of the delivery system of FIG. 1 during successive steps in the deployment of a prosthesis, wherein FIG. 2 depicts the step of advancing the delivery system along a guiding member and positioning the delivery system at a target site within a patient, FIG. 3 depicts the step of deploying the prosthesis and separating the guiding member from the delivery system, and FIG. 4 depicts the complete separation of the guiding member from the delivery system following deployment of the prosthesis;

FIG. 5 is a sectional side view of the distal portion of the inner member of an illustrative delivery system;

FIG. 6 is a cross-sectional view of the inner member taken along line 6-6 of FIG. 5;

FIGS. 7 and 7a are cross-sectional views of the inner member taken along line 7-7 of FIG. 5, wherein FIG. 7 depicts the channel of the inner member in a semi-closed first configuration and FIG. 7a depicts the channel in a fully open second configuration;

FIGS. 8 and 8a are cross-sectional views of the inner member taken along line 8-8 of FIG. 5, wherein FIG. 8 depicts the channel of the inner member in a semi-closed first configuration and FIG. 8a depicts the channel in a fully open second configuration;

FIGS. 9 and 10 illustrate an alternative embodiment of the delivery system having a projection extending outwardly from the inner member for engaging and separating the wall of the outer member, wherein the FIG. 9 depicts the delivery system prior to deployment of the prosthesis and FIG. 10 depicts the delivery system during deployment of the prosthesis;

FIG. 11 is a top view of the embodiment of the inner member illustrated in FIGS. 9 and 10 depicting the outwardly extending projection; and

FIG. 12 is a sectional side view of the embodiment of the inner member illustrated in FIGS. 9 and 10 depicting the outwardly extending projection.

### DETAILED DESCRIPTION

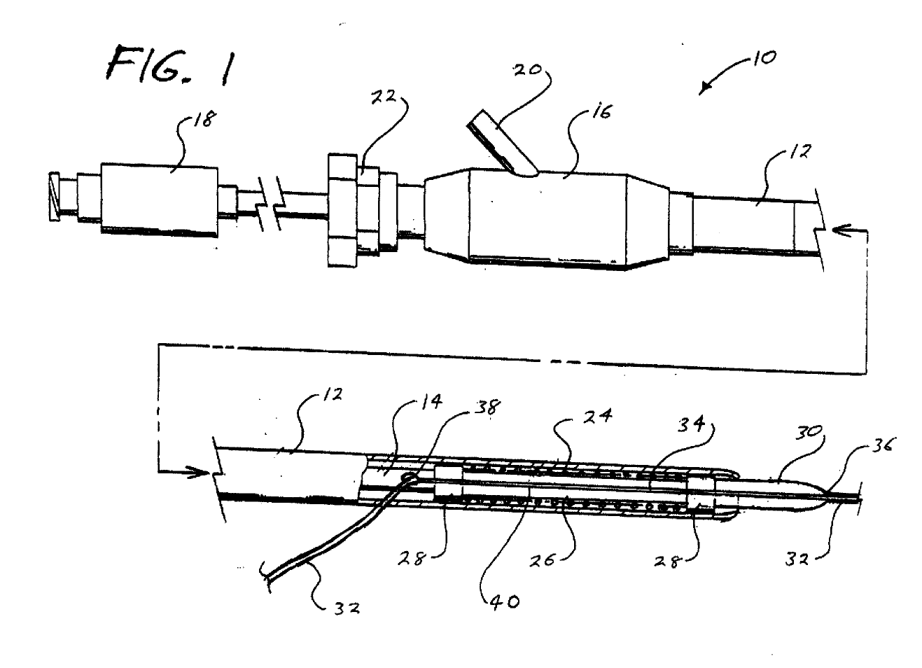

An exemplary prosthesis delivery system and method for introducing an expandable prosthesis over an indwelling guiding member, such as a wire guide, into a patient by remotely uncoupling the delivery system from the guiding member within the work site (defined as a lumen, duct, organ, vessel, other bodily passage or cavity, or the pathway leading thereto in which wire guide/guiding member access is maintained throughout a particular procedure or series of procedures), thereby facilitating the removal of the delivery system and simplifying introduction of a secondary access device or delivery system over the indwelling wire without an exchange of devices taking place outside of the patient is shown in the embodiments illustrated in FIGS. 1-12.

A first exemplary embodiment of the delivery system 10 is depicted in FIGS. 1-8a, which comprises an elongate outer member 12 that is slidably disposed about an elongate inner member 14. The elongate outer member 12 is commonly referred to as a sheath and the inner member 14 is commonly referred to as a pusher member. With reference to FIG. 1, a first handle portion 16 is affixed to the proximal end of the outer member 12 and a second handle portion 18 is affixed to the proximal end of the inner member 14. As will be explained in greater detail below, the first and second handle portions 16, 18 are configured to allow a user to longitudinally move the outer member 12 relative to the inner member 14 by grasping and moving the handle portions relative to each other. The first handle portion 16 includes a fluid access port 20 for introducing a fluid into the cavity between the outer and inner members 12, 14. For example, saline may be injected through the fluid access port 20 to flush air out of the interior of the delivery system 10 prior to introducing the system into the patient. A seal 22 is provided on the proximal end of the first handle portion 16 for preventing any fluids introduced via the fluid access port 20 or entering the distal end of the system from passing out through the proximal end of the first handle portion 16. It should be noted that many of the above-described features are optional, such as fluid access port 20 and seal 22. In addition, the delivery system 10 may include other features known by those skilled in the art of prosthesis delivery systems.

As best seen in FIG. 1, a self-expanding prosthesis 24 is disposed within the distal portion of the outer member 12. In the particular embodiment illustrated, the self-expanding prosthesis 24 comprises a self-expanding stent, such as the ZILVER® Stent sold by Cook®, Bloomington, Ind. However, any nitinol, stainless steel, or other self-expanding stent, artificial valve (e.g., venous, heart, pulmonary, etc.), prosthesis, vessel occluder, filter, embolic protection device, shunt, stent graft, etc. could be disposed within the outer member 12 of the delivery system 10. The prosthesis 24 is constrained in a compressed delivery configuration by the outer member 12 until it is deployed from the distal end thereof.

The prosthesis 24 is disposed about the inner member 14 and is generally constrained against longitudinal movement relative thereto. In particular, the inner member 14 includes a prosthesis carrier region 26 that is bounded at each end by a pusher band 28. The prosthesis carrier region 26 has a reduced diameter that forms a cavity between the exterior surface of the inner member 14 and the interior surface of the outer member 12 into which the prosthesis 24 is disposed. The pusher bands 28 have a relatively larger diameter that closely matches the interior diameter of the outer member 12, thereby enclosing the ends of the cavity of the prosthesis carrier region 26. The pusher bands 28 function to prevent longitudinal movement of the prosthesis 24 relative to the prosthesis carrier region 26 of the inner member 14. More specifically, the pusher bands 28 secure the prosthesis 24 against movement as the outer member 12 is retracted proximally relative to the inner member 14 during deployment of the prosthesis 24. In the embodiment illustrated, the pusher bands 28 comprise a radiopaque material to enable the user to fluoroscopically view the position of the distal portion of the delivery system 10, i.e., the prosthesis carrier region 26, inside the patient. The inner member 14 further comprises an end cap 30 having an atraumatic tip to facilitate advancement of the delivery system 10 through the internal vessels or ducts of the patient. The end cap 30 may be a separately formed component affixed to the distal end of the inner member 14, or may be integrally formed therewith.

The delivery system 10 is configured for attachment or coupling to an elongate guiding member, such as wire guide 32. In particular, the delivery system 10 includes a lumen or passageway 34 extending through at least a portion of the inner member 14. The passageway 34 extends between a distal opening or port 36 disposed in the distal end of the end cap 30 and a proximal opening or port 38 in the inner member 14. In the particular embodiment illustrated in FIG. 1, the proximal opening 38 is located a short distance proximal to the prosthesis carrier region 26. Thus, the passageway 34 extends through the prosthesis carrier region 26. The proximal opening 38 may, however, be located at other locations, such as through the side of the end cap 30 just proximal of the distal opening 36 to provide a very short passageway 34, or may be spaced a greater distance proximally of the prosthesis carrier region 26 to provide a relatively long passageway 34. The inner member 14 may further comprise a channel 40 through the side wall thereof that is in communication with the passageway 34. As will be explained in greater detail below, the channel 40 provides a pathway for the wire guide 32 to laterally pass out of the passageway 34.

The outer member 12 similarly includes an opening 42 through the side wall thereof. The opening 42 in the outer member 12, which is best seen in FIG. 2, is in communication with the proximal opening 38 of the inner member 14. More specifically, the opening 42 in the outer member 12 is configured to overlie the proximal opening 38 in the inner member 14 when the outer and inner members 12, 14 are positioned in the initial or delivery configuration.

The passageway 34 and the openings 36, 38, 42 in the inner and outer members 12, 14 are each configured for the passage of a guiding member, such as wire guide 32, therethrough and collectively form a coupling region. In particular, and as best seen in FIG. 1, the delivery system 10 may be coupled to a wire guide 32 by passing or back-loading the wire guide 32 through distal opening 36, passageway 34, and proximal opening 38 of the inner member 14, and then through opening 42 of the outer member 14 (see FIG. 2). Thus, the delivery system 10 may be coupled to and advanced along a guiding member, such as wire guide 32, which has been previously positioned within the patient. The delivery system 10 and the guiding member may also be coupled together and simultaneously advanced into the work site in a coupled state. Since the passageway 34 is substantially shorter in length than the overall length of the delivery system 10, a substantial portion of the guiding member (i.e., the portion extending proximally of opening 40) is disposed outside of the delivery system 10, thereby giving the user or physician more control over the guiding member. In the particular embodiment illustrated, the guiding member comprises either a 0.018 or 0.035 diameter ROADRUNNERS Wire Guide, sold by Cook®), Bloomington, Ind., or a 0.035 diameter TRACER METRO DIRECT™ wire guide, sold by Cook® Endoscopy, Winston-Salem, N.C. However, any other guiding device suitable for coupling to and guiding the delivery system 10 to the target or work site within the patient may be employed.

For purposes of this disclosure, the work site is defined as the lumen, duct, organ, vessel, or other bodily passage/cavity, or the pathway leading thereto, in which wire guide access is maintained to perform a particular medical procedure/operation or series of procedures. For example, in a procedure involving the vasculature system, the work site may be the carotid artery and the vascular ducts extending therefrom. Similarly, in a procedure involving the biliary system, the work site is typically the common bile duct, including the pancreatic duct and the ducts extending into the lobes of the liver.

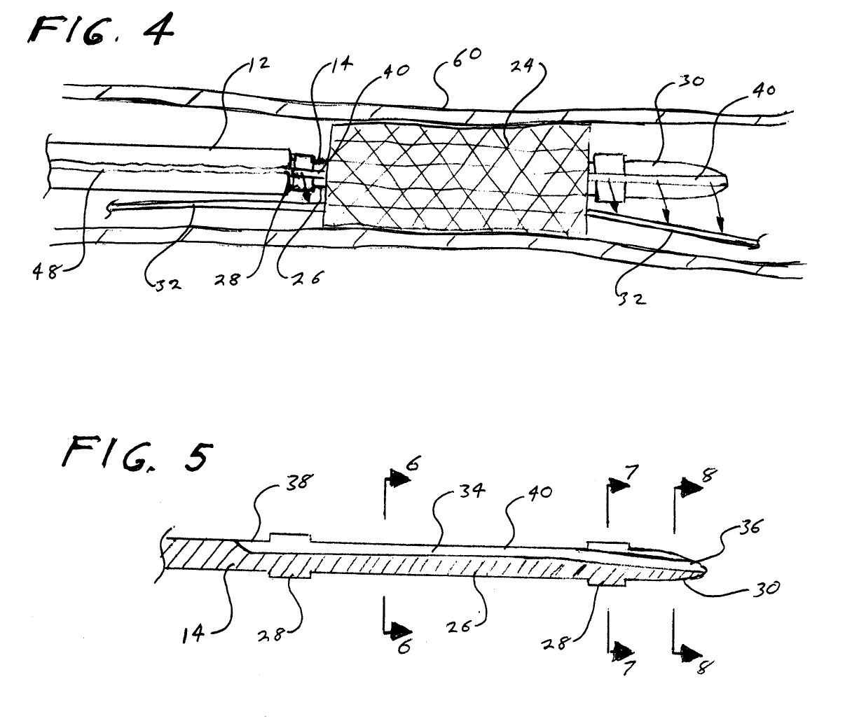

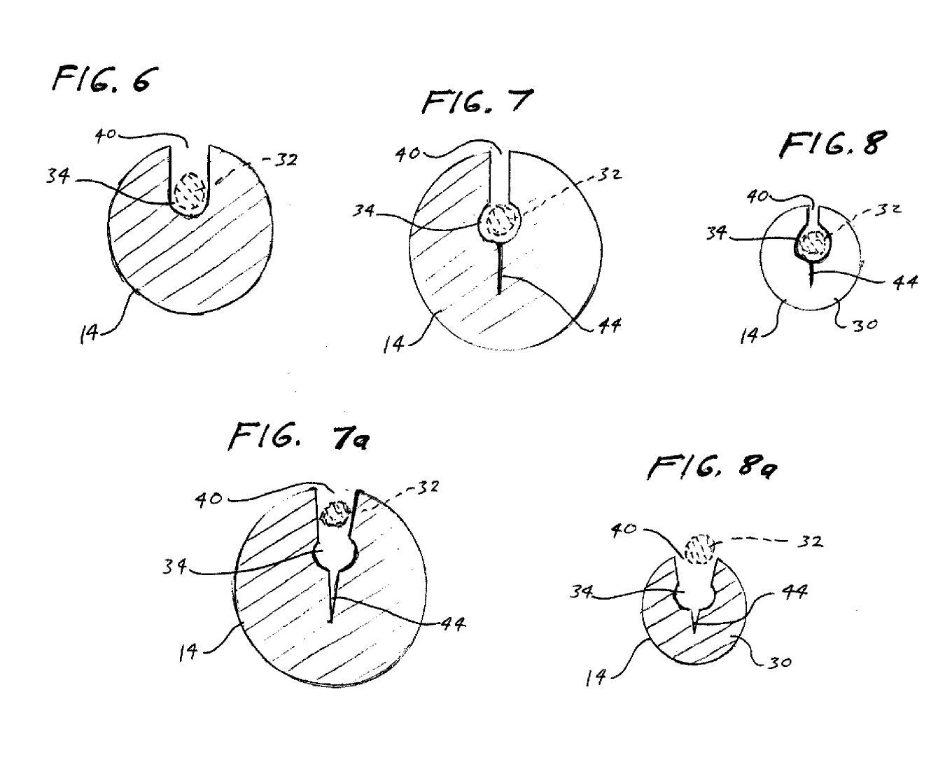

As mentioned above, the inner member 14 may further comprise a channel 40 through the side wall thereof that is in communication with the passageway 34. The channel 40 provides a pathway for the wire guide 32 to laterally pass out of the passageway 34 once the outer member 12 has been proximally retracted (relative to the inner member 14) to deploy the prosthesis 24. As best seen in FIGS. 5-8a, the channel 40 comprises different dimensions at different locations along the length thereof. In addition, at least a portion of the channel 40 is moveable between a first configuration and a second configuration. For example, and as illustrated in FIGS. 7 and 8, the channel 40 comprises a first configuration having a width that is less than the diameter of the guiding member, such as the wire guide 32 shown in phantom lines. When the channel 40 is in this first configuration, the wire guide 32 is prevented from laterally passing out through the channel 40, thereby containing the wire guide 32 within the passageway 34 of the inner member 14. However, and as illustrated in FIGS. 7a and 8a, the channel 40 may further comprise a second configuration having a width that is greater than the diameter of the guiding member (wire guide 32). When the channel 40 is in this second configuration, the wire guide 32 is allowed to laterally pass out through the channel 40, thereby permitting the wire guide 32 to be laterally removed from the passageway 34 and uncoupled from the inner member 14.

In the embodiment illustrated, the inner member 14 is manufactured or other wise biased to have the cross-section shown in FIGS. 7a and 8a, i.e., with the channel 40 disposed in the fully open second configuration. When assembled with the outer member 12 (see FIG. 1), the inner member 14 is compressed so as to move the channel 14 into the partially closed first configuration shown in FIGS. 7 and 8. Thus, and as will be explained below in connection with a description of the prosthesis deployment procedure, the channel 40 will revert to the fully open second configuration when the outer member 12 is proximally retracted a distance sufficient to remove the compressive force on the inner member 14. A slit 44 may be formed in the wall of the inner member 14 opposite the channel 40 to facilitate and increase movement of the channel between the first and second configurations.

In the particular embodiment illustrated, the portion of the channel 40 that is moveable between a first and second configuration is generally limited to the end cap 30. More specifically, and as illustrated in FIG. 6, the portion of the channel 40 within the prosthesis carrier region 26 has a width greater than the diameter of the guiding member (wire guide 32) and is not moveable to a closed position having a smaller width (i.e., the channel 40 has the same width as the passageway 34). This is because the wire guide 32 will be contained within the passageway 34 and/or channel 40 by the prosthesis 24 along this portion of the inner member 14. However, it should be understood that this portion of the channel 40 could similarly be made movable to prevent the wire guide 32 from coming into contact with the prosthesis 24.

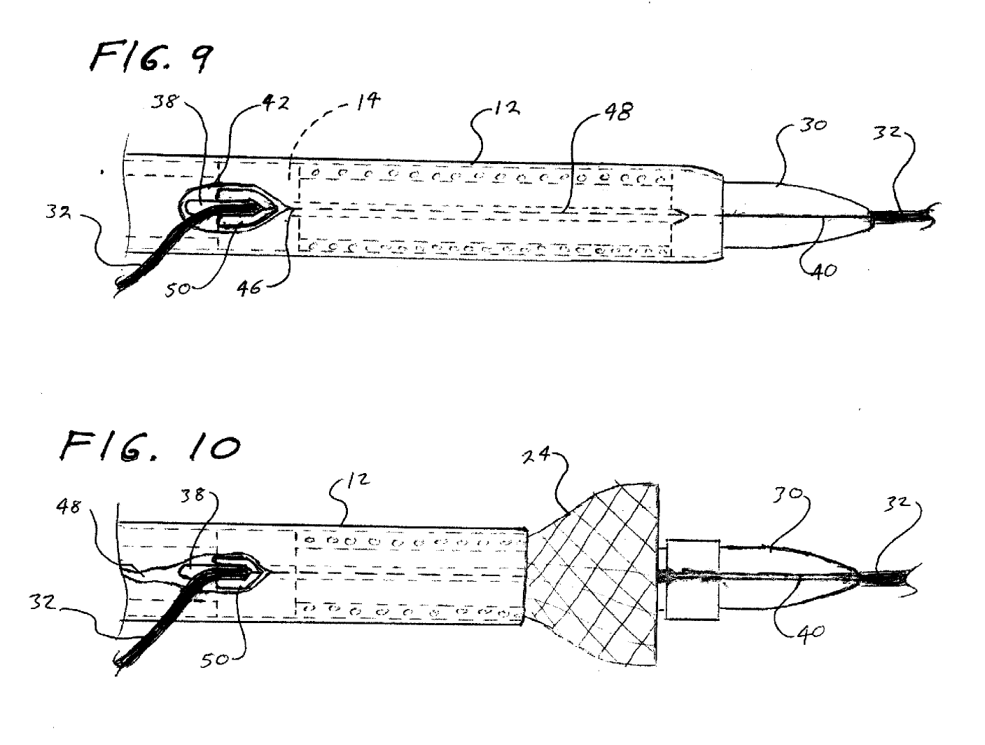

The channel 40 may comprise any number of configurations capable of moving from a first configuration that will contain the guiding member within the passageway 34 prior to deployment of the prosthesis 24 to a second configuration that will allow the lateral removal of the guiding member from the passageway 34 upon deployment of the prosthesis 24. For example, the channel 40 may comprise a partially closed configuration when in the first configuration as depicted in FIGS. 7 and 8. However, the channel 40 may alternatively comprise a fully closed configuration when in the first configuration, as shown in FIGS. 9-10 with respect to the end cap 30. FIG. 9 shows the channel 40 of the end cap 30 in a fully closed initial state and FIG. 10 shows the channel 40 slightly open as the outer member 12 is partially retracted. A fully closed configuration may be advantageous in preventing the passageway 34 from becoming occluded with bodily fluids during advancement of the delivery system 10. However, a fully closed configuration requires a greater expansion of the channel opening that will be sufficient to allow a guiding member to pass therethrough when in the second configuration.

The location of the passageway 34 relative to the exterior surface of the inner member 14 similarly changes along the length thereof. For example, and as illustrated in FIG. 6, the passageway 34 may be offset from the center of the inner member 14 so as to reduce the depth of the channel 40. Positioning the passageway 34 near to the exterior surface of the inner member 14 increases the likelihood that the guiding member (wire guide 32) will be expelled and become uncoupled therefrom during the prosthesis deployment procedure. However, and as illustrated in FIGS. 5 and 8, it is preferable to dispose the passageway 34 closer to the center of the inner member 14 as the passageway 34 nears the distal opening 36, thereby allowing the distal most portion of the delivery system 10 to be centered about the guiding member. Such a configuration is less likely to cause the delivery system 10 to snag or get caught on the interior surface of the bodily lumen as it is advanced into the patient. In addition, and as best seen in FIG. 5, the passageway 34 preferably has a generally curvilinear axis that is bowed outwardly relative to the central axis of the inner member 14 along a portion thereof. Such a profile will tend to ‘spring’ the guiding member out of the passageway 34 when no longer contained therein by the closed or partially closed channel 40 or the prosthesis 24. In the particular embodiment illustrated, the ‘bow’ or bend in the passageway 34 is located within the prosthesis carrier region 26 just proximal to the distal pusher band 28. The passageway 34 may also include a separate biasing member (not shown) such as an elastic pad or a metal leaf spring disposed in the bottom thereof which is configure to expel the guiding member therefrom.

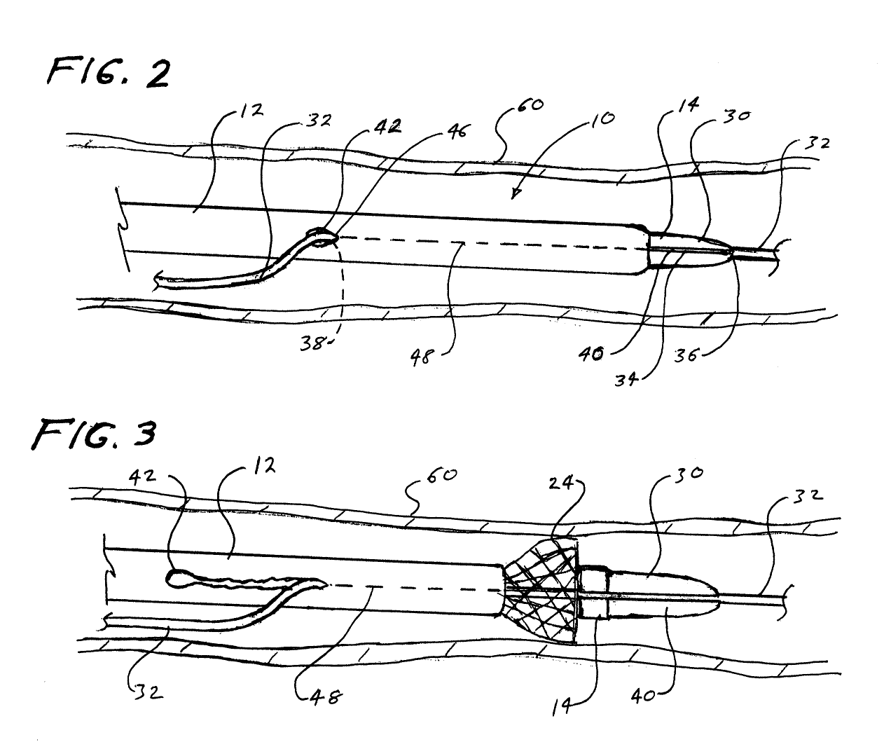

As explained above, the outer member 12 comprises an opening 42 through the side wall thereof. As best seen in FIG. 2, the opening 42 overlies the proximal opening 38 of the inner member 14 when the delivery system 10 is in the delivery configuration. This arrangement allows a guiding member, e.g. wire guide 32, to exit the proximal opening 38 of the inner member 14 and pass out through the opening 42 of the outer member 12. In the particular embodiment illustrated, the opening 42 comprises a teardrop shape having a relatively sharp inner corner 46 at the distal end thereof. As will be explained in greater detail below, the relatively sharp inner corner 46 facilitates the rupture or separation of the outer member 12 along tear-line 48 during retraction of the outer member 12 relative to the inner member 14 during deployment of the prosthesis 26. More specifically, and as shown in FIG. 3, the outer member 12 is configured to separate or split along tear-line 48 so as to pass around the portion of the wire guide 32 exiting the proximal opening 38 of the inner member 14. This allows the outer member 12 to be separated from the wire guide 32 (see FIG. 4) without the need to retract the wire guide 32 through the opening 42, thereby allowing the wire guide 32 to remain stationary during the prosthesis deployment procedure.

In the particular embodiment illustrated in FIGS. 1-4, the tear-line 48 comprises a portion of the outer member 12 that may be ruptured or split as it is engaged by the wire guide 32 as the outer member is retracted in a proximal direction relative to the inner member 14. The tear-line 48 may comprise a portion of the outer member 12 that has been weakened by, for example, a groove or perforation extending through the wall thereof. The tear-line 48 may also comprise a material that has a relatively low resistance to rupture. Alternatively, the tear-line 48 may comprise an open slot having a width greater than the diameter of the wire guide 32 to allow the outer member 12 to pass by the wire guide 32 without needing to spread apart. However, in this alternative embodiment, the portion of the outer member 12 that encloses the prosthesis carrier region 26 of the inner member 14 (i.e., the distal most portion of the outer member 12) must be relatively rigid to contain the prosthesis 24 in the compressed delivery configuration irrespective of the slot through the wall thereof. For example, the outer member 12 may include reinforcing members, such as a plurality of C-shaped metal coils, that extend about the circumference of the wall, but which do not transverse the open slot of the tear-line 48. The metal coils maintain the cross-sectional shape of the outer member 12 and prevents the prosthesis from spreading the outer member 12 outwardly.

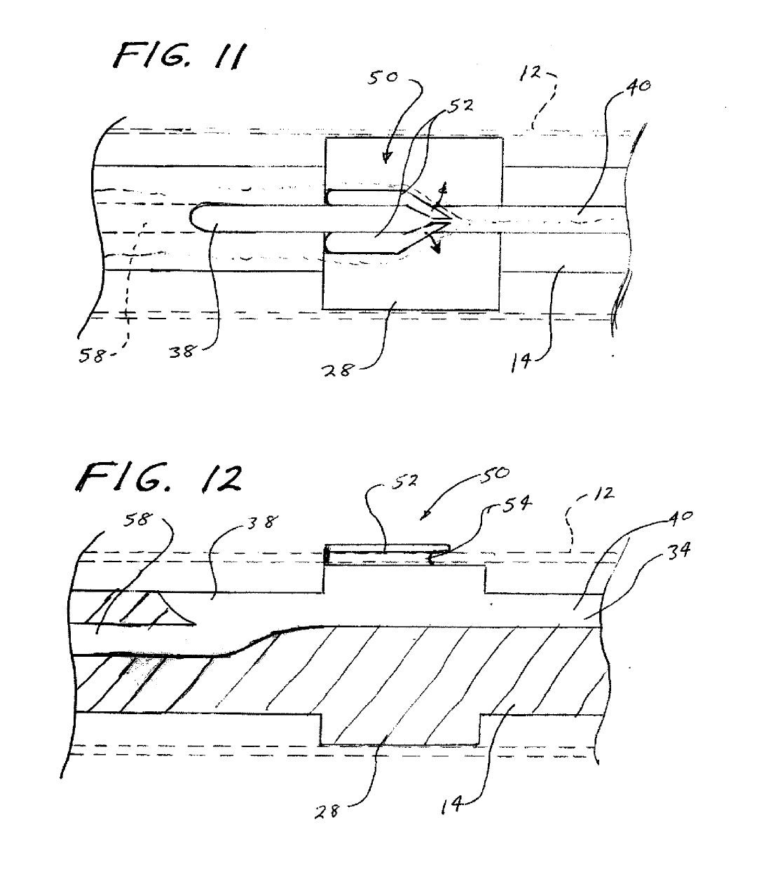

FIGS. 9-12 illustrate an alternative embodiment of the delivery system 10 having a separating member 50 extending outwardly from the inner member 14 for engaging and separating the wall of the outer member 12 along tear-line 48. FIG. 9 depicts the distal portion of the delivery system 10 prior to deployment of the prosthesis 24 and FIG. 10 depicts the delivery system 10 during deployment of the prosthesis 24. FIG. 11 is a top view and FIG. 12 is a sectional side view of the separating member 50 and the inner member 14, with the outer member 12 shown in phantom lines for clarity. In the particular embodiment illustrated, the separating member 50 comprises a pair of teeth 52 affixed to the exterior surface of the proximal pusher band 28 that extend through the opening 38 of the outer member 12. As best seen in FIG. 12, the teeth 52 each include a cutting edge 54 configured to engage and sever the wall of the outer member 12 as the outer member 12 is retracted in a proximal direction relative to the inner member 14 (see FIG. 10). The teeth 52, and more specifically the cutting edges 54, reduce or eliminate the need to provide a weakened area in the outer member 12 along tear-line 48. The teeth 52 also prevent the outer member 12 from engaging and possibly dislodging the position of the wire guide 32 as the outer member 12 is retracted during deployment of the prosthesis 24. As best seen in FIG. 11, the pair of teeth 52 spread apart from each other once the distal end of the outer member 12 has moved completely past the separating member 50. This allows the wire guide 32 to pass laterally out of the passageway 34, the channel 40 and past the teeth 52 so as to separate/uncouple from the inner member 14. As best seen in FIG. 12, each of the teeth 52 may also include a guard 56 to prevent the cutting edges 54 from engaging and possibly damaging the bodily lumen as the delivery system 10 is advanced therethrough.

In the particular embodiment illustrated in FIGS. 9-12, the separating member 50 comprises a pair of flexible teeth 52. However, only a single tooth 52 could be employed. Likewise, the separating member 50 may comprise one or more rigid teeth 52. If rigid teeth 52 are employed, then the teeth 52 should be positioned so as to not prevent the wire guide 32 from laterally exiting the channel 40 of the inner member 14. For example, a pair of rigid teeth 52 may be positioned along either side of the channel 40 in a spaced apart configuration so as to not infer with lateral movement of the wire guide 32 into and out of the passageway 34. It should be appreciated that the separating member 50 may comprise any number of shapes or configurations that is capable of severing or splitting the wall of the outer member 12 along tear-line 48.

The embodiment illustrated in FIGS. 11 and 12 also comprises a proximal lumen 58 extending through the inner member 14 proximally of the proximal opening 38. As will be explained in greater detail below, the proximal lumen 58 can be employed to introduce a second guiding member into the patient after the first guiding member has been uncoupled from the delivery system 10. This may be advantageous, for example, when performing individual procedures in each leg of a bifurcated duct.

An exemplary method of delivering a self-expanding prosthesis 24 to a work site within the lumen of a patient, employing the prosthesis delivery system 10 of the present invention, will be described in connection with FIG. 24. As shown in FIG. 2, the distal portion of the delivery system 10 is first positioned at the target site within the patient's bodily lumen 60. For example, the prosthesis 24, which is compressed within the outer member 12, may be positioned so as to span a stricture in the lumen 60. This is accomplished by advancing the distal end of the delivery system 10 over a guiding member, such as wire guide 32, which has been previously positioned within the lumen 60. More specifically, the proximal end of the wire guide 32 is inserted or back-loaded through distal opening 36, passageway 34, and proximal opening 38 of the inner member 14, and then through opening 42 of the outer member 14. The delivery system 10 is then pushed is a distal direction along wire guide 32 until it reaches the target or work site within the patient. Fluoroscopy may also be employed to aid in the proper positioning of the wire guide 32 and the delivery system 10.

As shown in FIG. 3, the outer member 12 is then retracted in a proximal direction relative to the inner member 14 to initiate the deployment procedure. Since the prosthesis 24 is restrained against movement relative to the inner member 14 by proximal pusher band 28 (see FIG. 1), the prosthesis 24 is forced out of the distal end of the outer member 12. The portion of the prosthesis 24 that is no longer contained within the outer member 12 is allowed to self-expand to its expanded configuration, whereby it engages the interior wall of the lumen 60. As the outer member 12 is retracted, the portion of the outer member 60 distally adjacent to the opening 42 engages and is split by the portion of the wire guide 32 exiting from the inner member 14 through proximal opening 38. This results in severing or splitting of the outer member 14 along tear-line 48, thereby allowing the outer member 12 to move proximally past the wire guide 32. In addition, as the outer member 12 is retracted, the distal most portion of the inner member 14 (e.g., cap 30) is allowed to expand so as to widen the gap of the channel 40. As a result, the wire guide 32 is no longer constrained within the passageway 34 along this portion of the inner member 14, which thereby allows the wire guide 32 to laterally separate from the inner member 14.

As shown in FIG. 4, the outer member 12 is further retracted in a proximal direction until the distal end thereof is moved completely proximal to the prosthesis carrier region 26 and the proximal pusher band 28. This action allows the prosthesis 24 to assume its final expanded configuration within the lumen 60. In addition, this action allows the wire guide 32 to move laterally out of the passageway 34 and through the channel 40, thereby allowing the wire guide 32 to uncouple/disengage from the inner member 14. The wire guide 32 likewise becomes uncoupled/disengaged from the outer member 12 once the distal end of the outer member 12 moves proximally past the proximal opening 38 of the inner member 14.

In the event that the wire guide 32 does not fully uncouple/disengage from the inner member 14 once the outer member 12 has been retracted as described above, then the outer member 12 can be utilized to force the wire guide 32 out of the passageway 34 and through the channel 40. More specifically, the outer member 12 can be retracted until the distal end thereof is proximal of the portion of the wire guide 32 extending out of the inner member 14. The outer member 12 is then rotated so as to offset the tear-line 48 from the channel 40 (and the wire guide 32). The outer member 12 may then be advanced so as to engage the wire guide 32 and push the wire guide 32 out of the passageway 34 and channel 40.

Once the wire guide 32 becomes fully uncoupled from the delivery system 10, the delivery system 10 can be removed from the patient. The wire guide 32 can likewise be removed from the patient, or may be maintained with the lumen 60 of the patient and utilized for the introduction of a second prosthesis delivery device or other type of elongate medical device. For example, the wire guide 32 can be utilized to introduce a balloon catheter device, which may be used to “set” the prosthesis 24 against the wall of the lumen 60.

This may be especially advantageous in deployment of stents, other prostheses, and other ancillary devices, such as dilation balloons, within the vascular system in that recannulation through the deployed stent may be problematic, possibly leading to complications such as dislodgement or catching on the deployed stent, dislodgement of plaque, etc. With regard to placement of artificial venous and other types of artificial valves, maintaining wire guide access through the valve may be particularly advantageous in that recannulation through the leaflets or valve structure to deploy additional valves or introduce a seating balloon to fully expand the valve support frame against the walls of the vessel may prove particularly difficult, possibly leading to damage of delicate leaf structure and compromise of valve function.

Once the wire guide 32 becomes fully uncoupled from the delivery system 10, the delivery system 10 can also be utilized to introduce a second guiding member. More specifically, the embodiment of the delivery system 10 illustrated in FIGS. 11 and 12 can be used to introduce a second wire guide by advancing the second wire guide through the proximal lumen 58 until it exits the inner member 14 near proximal opening 38. Once the second wire guide has been introduced into the patient, the inner member 14 can then be retracted and withdrawn from the patient. This may be particularly advantageous in procedures that require the simultaneous placement of two separate wire guides within the patient. For example, when placing a stent in each leg of a bifurcated duct, a separate wire guide can be positioned in each leg, thereby allowing two delivery systems to be introduced into the patient without needing to re-position a singe wire guide from one leg to the other.

Preferably, both the outer member 12 and the inner member 14 comprise a material having a sufficient lateral flexibility and longitudinal rigidity to facilitate the introduction of the delivery system 10 into the patient. This especially advantageous in long delivery systems such as those used to deploy carotid artery stents, biliary stents, venous or other artificial valves, etc. For example, the outer member 12 may comprise a sheath with a superior combination of flexibility and rigidity characteristics, such as Cook's FLEXOR® Sheath or C-FLEX® stent material (Cook Incorporated, Bloomington, Ind.), while the inner member 14 may include a coiled wire with a polyamide sheath attached thereto. In the embodiment illustrated, the inner member 14 comprises different portions having different properties, wherein the distal portion comprises a typical catheter material, such as PEEK, that is not particularly rigid, while the proximal section comprises a more rigid portion, such as the above-described coiled sheath.

Any other undisclosed or incidental details of the construction or composition of the various elements of the disclosed embodiment of the present invention or methods of their use are not believed to be critical to the achievement of the advantages of the present invention, so long as the elements possess the attributes needed for them to perform as disclosed. The selection of these and other details of construction are believed to be well within the ability of one of even rudimentary skills in this area, in view of the present disclosure. Illustrative embodiments of the present invention have been described in considerable detail for the purpose of disclosing a practical, operative structure whereby the invention may be practiced advantageously. The designs and methods described herein are intended to be exemplary only. The novel characteristics of the invention may be incorporated in other structural forms without departing from the spirit and scope of the invention. The invention encompasses embodiments both comprising and consisting of the elements and steps described with reference to the illustrative embodiments. Unless otherwise indicated, all ordinary words and terms used herein shall take their customary meaning as defined in The New Shorter Oxford English Dictionary, 1993 edition. All technical terms shall take on their customary meaning as established by the appropriate technical discipline utilized by those normally skilled in that particular art area. All medical terms shall take their meaning as defined by Stedman's Medical Dictionary, 27th edition.

What is claimed is:

1. A delivery system for delivering a self-expanding prosthesis to a target location within a bodily lumen of a patient comprising:
an elongate inner member extending between a distal end and a proximal end, the inner member comprising a passageway extending through at least a portion thereof between a distal opening disposed near the distal end and a proximal opening spaced proximally from the distal opening, the passageway comprising a coupling region for coupling to a guiding member;
an elongate outer member slidably disposed about the inner member, the outer member comprising an opening in communication with the proximal opening of the inner member; and
a self-expanding prosthesis disposed about the inner member and within the outer member;
wherein the prosthesis is deployable from the delivery system by proximal movement of the outer member relative to the inner member; and
wherein a guiding member disposed within the passageway of the inner member is laterally separatable from the inner member by proximal movement of the outer member relative to the inner member to thereby create an open channel for the guiding member to exit therefrom.

2. The delivery system of claim 1, wherein a guiding member extending through the opening of the outer member is separable from the outer member by proximal movement of the outer member relative to the inner member.

3. The delivery system of claim 2, wherein a portion of the outer member adjacent to the opening is adapted to separate as a result of proximal movement of the outer member relative to the inner member.

4. The delivery system of claim 3, wherein the portion of the outer member adjacent to the opening comprises a wall having one of a slot, a groove, a perforation and a weakened material.

5. The delivery system of claim 3, wherein the opening comprises a distal edge portion having a reduced radius to facilitate separation of the outer member as a result of proximal movement of the outer member relative to the inner member.

6. The delivery system of claim 5, wherein the opening comprises a teardrop shape.

7. The delivery system of claim 3, wherein the portion of the outer member adjacent to the opening is separated by engagement with a portion of the guiding member extending out of the proximal opening of the inner member.

8. The delivery system of claim 3, wherein the inner member comprises an outwardly extending separating member for engaging the portion of the outer member adjacent to the opening upon proximal movement of the outer member relative to the inner member.

9. The delivery system of claim 8, wherein the separating member comprises at least one tooth configured for severing the portion of the outer member adjacent to the opening upon proximal movement of the outer member relative to the inner member.

10. The delivery system of claim 8, wherein the separating member comprises a pair of flexible teeth configured for severing the portion of the outer member adjacent to the opening upon proximal movement of the outer member relative to the inner member, the pair of flexible teeth being movable between a first position wherein the teeth are substantially touching each other, and a second position wherein the teeth are spaced apart from each other a distance sufficient to allow the guiding member to laterally pass therebetween.

11. The delivery system of claim 8, wherein the separating member is disposed within the opening of the outer member.

12. The delivery system of claim 1, wherein a guiding member extending through the passageway of the inner member is separable from the inner member by proximal movement of the outer member relative to the inner member.

13. The delivery system of claim 12, wherein the inner member comprises a channel in communication with the passageway, at least a portion of the channel being movable between a first configuration wherein the guiding member is laterally confined within the passageway, and a second configuration wherein the guiding member is not laterally confined within the passageway.

14. The delivery system of claim 13, wherein the movable portion of the channel moves from the first configuration to the second configuration as a result of proximal movement of the outer member relative to the inner member.

15. The delivery system of claim 13, wherein the movable portion of the channel comprises a first width that is less than a diameter of the guiding member when in the first configuration and a second width that is greater than the diameter of the guiding member when in the second configuration.

16. The delivery system of claim 13, wherein a distal portion of the inner member comprises a tapered end cap, and further wherein the movable portion of channel is disposed along the end cap.

17. The delivery system of claim 13, wherein the inner member comprises a wall portion adjacent to the passageway and opposite of the movable portion of the channel, the wall portion having a slit to facilitate movement of the channel between the first and second configurations.

18. The delivery system of claim 12, wherein the passageway comprises a non-linear central axis that is configured to facilitate lateral removal of the guiding member therefrom.

19. The delivery system of claim 17, wherein the central axis of the passageway is offset from a central axis of the inner member along at least a portion thereof.

20. A method of a self-expanding prosthesis to a target location within a bodily lumen of a patient comprising:
providing a prosthesis delivery system comprising an elongate outer member slidably disposed about an elongate inner member, and further comprising a self-expanding prosthesis disposed about the inner member and within the outer member, the inner member comprising a passageway extending through at least a portion thereof between a distal opening and a proximal opening spaced proximally from the distal opening, the outer member comprising an opening in communication with the proximal opening of the inner member;
advancing the prosthesis delivery system along a guiding member, the guiding member extending through the passageway and openings of the inner and outer members,
positioning a distal portion of the prosthesis delivery system at the target location within the patient;
retracting the outer member in a proximal direction relative to the inner member to simultaneously deploy the prosthesis from the delivery system and laterally separate the guiding member from the inner member.

21. The method of claim 20, wherein the step of retracting the outer member in a proximal direction relative to the inner member to simultaneously deploy the prosthesis from the delivery system and laterally separate the guiding member from the inner member is accomplished while maintaining the longitudinal position of the guiding member relative to the inner member.

22. The method of claim 20, wherein the step of retracting the outer member in a proximal direction relative to the inner member to simultaneously deploy the prosthesis from the delivery system and laterally separate the guiding member from the inner member also causes the guiding member to separate from the outer member.
